(19) United States
(12) Patent Application Publication
(10) Pub. No.: US 2006/0281702 A1
(43) Pub. Date: Dec. 14, 2006

(54) Combinatorial selection of phosphorothioate aptamers for RNases

(75) Inventor: David G. Gorenstein, Houston, TX (US)

Correspondence Address: CHALKER FLORES, LLP, 2711 LBJ FRWY, Suite 1036, DALLAS, TX, 75234, US

(73) Assignee: Board of Regents, The University Of Texas System, Austin, TX (US)

(21) Appl. No.: 11/435,907

(22) Filed: May 17, 2006

Related U.S. Application Data
(60) Provisional application No. 60/682,287, filed on May 18, 2005.

(51) Int. Cl. A61K 48/00; C40B 40/08; C40B 50/06

(52) U.S. Cl. 514/44; 435/6

(57) ABSTRACT

The present invention includes the selection and isolation of thioaptamers that target the ribonuclease domains of enzymes, e.g., HIV reverse transcriptase.

### TECHNICAL FIELD OF THE INVENTION

The present invention relates in general to the field of partially thio-modified aptamers or thioaptamers, and more particularly, to thioaptamers that target RNases and the development of therapeutic agents based thereon.

### STATEMENT OF FEDERALLY FUNDED RESEARCH

This work was supported by the following United States grants: NIH AI27744—Combinatorial and rational design of aptamers targeting HIV, NHLBI N01-HV-28184—Proteomic Technologies to Study Airway Inflammation; and NIAID U01 AI054827—Biodefense Proteomics Collaboratory, the government may own certain rights.

### BACKGROUND OF THE INVENTION

This application claims priority to U.S. Provisional Patent Application Ser. No. 60/682,287, filed May 18, 2005, the entire contents of which are incorporated herein by reference. Without limiting the scope of the invention, its background is described in connection with oligonucleotide agents and with methods for the isolation of modified oligonucleotides that bind specifically to target proteins.

Virtually all organisms have nuclease enzymes that degrade rapidly foreign DNA as an important in vivo defense mechanism. The use, therefore, of normal oligonucleotides or aptamers as diagnostic or therapeutic agents in the presence of most bodily fluids or tissue samples is generally precluded. It has been shown, however, that phosphoromonothioate or phosphorodithioate modifications of the DNA backbone in oligonucleotides can impart both nuclease resistance and enhance the affinity for target molecules, such as for example the transcriptional regulating protein NF-κB. Therefore, there is a need in the art for methods for generating aptamers that have enhanced binding affinity for a target molecule, as well as retained specificity. Also needed are ways to identify and quantify in detail the mechanisms by which aptamers interact with target molecules.

Synthetic phosphodiesier-modified oligonucleotides such as phosphorothioate oligonucleotide (S—ODN) and phosphorodithioate oligonucleotide (S₂—ODN) analogues have increased nuclease resistance and may bind to proteins with enhanced affinity. Unfortunately, ODNs possessing high fractions of phosphorothioate or phosphorodithioate linkages may lose some of their specificity and are “stickier” towards proteins in general than normal phosphate esters, an effect often attributed to non-specific interactions. The recognition of nucleic acid sequences by proteins involves specific side-chain and backbone interactions with both the nucleic acid bases as well as the phosphate ester backbone, effects which may be disrupted by the non-specific interactions caused with S—ODN and S₂—ODN analogues.

### SUMMARY OF THE INVENTION

The present invention comprises a partially thio-modified aptamer that binds to a protein comprising RNase H activity. The aptamer may include the thioate and sequence substitutions of the oligonucleotides identified by SEQ ID NOS.:1, 2 and the combination thereof. For example, the aptamer may include the formula (wherein s is a thioate and/or a dithioate substitution):

| | |
|---|---|
| 5′ ATGCTTCCACGAGCCTTTCGGGGTTGGTGTsAC | (SEQ ID NO.:1) |
| sAGTGGsATGGCTGCGsAGGCGGTsAGTCTsATTC | |
| 3′ | |
| 3′ TAsCGAsAsGGTGCTCGGAsAsAsGCCCCAsAs | (SEQ ID NO.:2) |
| CCAsCAsTGTCAsCCTAsCCGACGCTCCGCCATCAG | |
| ATAAG 5′. | |

The aptamer may be resuspended and provided along with one or more pharmaceutically acceptable salts and/or a diluent. In one embodiment, the aptamer is achiral. The partially thio-modified aptamer may specifically bind to a Reverse Transcriptase (RT) that includes an RNase H domain, e.g., an HIV Reverse Transcriptase. The partially thio-modified aptamer may inhibits the Reverse Transcriptase activity of a retroviral RT, e.g., an HIV Reverse Transcriptase. The thio-modified aptamer may include one or more thio-modifications as set forth in SEQ ID NOS.: 1-32, and may even include a short interfering RNA (siRNA); a micro, interfering RNA (miRNA); a small, temporal RNA (stRNA); or a short, hairpin RNA (shRNA).

The present invention also includes a method of identifying an RNase H specific-thioaptamer by synthesizing a random phosphodiester oligonucleotide combinatorial library; contacting the partially thiophosphate-modified oligonucleotide combinatorial library with a protein having RNaseH activity; and isolating a subset of oligonucleotides binding to the target molecule. The one or more thio-modifications may be selected from the group consisting of dATP(αS), dTTP(αS), dCTP(αS), dGTP(αS), rUTP (αS), rATP(αS), rCTP(αS), rGTP(αS), dATP(αS₂), dTTP(αS₂), dCTP(αS₂), dGTP(αS₂), rATP(αS₂), rCTP(αS₂), rGTP(αS₂) and rUTP(αS₂), mixtures or combinations thereof. Generally, no more than three adjacent phosphate sites are replaced with phosphorothioate groups. In one embodiment, at least a portion of non-adjacent phosphate sites are replaced with phosphorothioate groups, e.g., no more than three adjacent phosphate sites are replaced with phosphorodithioate groups. In one example, the target protein is a viral reverse transcriptase, e.g., an HIV RT.

Another method of the present invention includes identifying a set of aptamers containing an optimal composition of thiophosphate-modified nucleotides such that the aptamers bind with high affinity to a protein having RNase H activity, and have increased resistance to nuclease degradation by synthesizing a random partially thiophosphate-modified oligonucleotide combinatorial library wherein at least a portion of the oligonucleotide phosphate groups are thiophosphate-modified nucleotides, and where no more than three of the four different nucleotides are substituted on the 5′-phosphate positions by 5′-thiophosphates in each synthesized oligonucleotide are thiophosphate-modified nucleotides; contacting the amplified library with the protein under conditions favorable for binding of a binding oligonucleotide with said target molecule; and isolating a subset of binding oligonucleotides from the library, that bind with higher affinity to the protein relative to the original library. The steps of the method may also be repeated selection iteratively, whereby an enriched subset of oligonucleotides binding with higher affinity to the target molecule relative to the original amplified subset, is isolated after each cycle. For example, each iteration may be performed under conditions of increased stringency in the contacting step until a subset of high affinity binding oligonucleotides is identified. The synthesis of the combinatorial library may be accomplished, in one example, using constituent oligonucleotides having at least a set of 5′ and 3′ PCR primer nucleotide sequences flanking a randomized nucleotide sequence. The subset of amplified oligonucleotides may also be cloned and individual thiophosphate-modified oligonucleotides that bind to the target isolated and sequenced.

Another embodiment of the present invention is a pharmaceutical formulation in which a therapeutically effective amount of a thioaptamer that bind specifically to and inhibits RNase H activity is provided to a patient in need thereof. The thioaptamer may be packed into a capsule, caplet, softgel, gelcap, suppository, film, granule, gum, insert, pastille, pellet, troche, lozenge, disk, poultice or wafer. The thioaptamer may be packaged for released within about 60 minutes or even release of over 90% within about 60 minutes and 12 hours. When provided in dry form, the formulation may be packaged with one or more of the following: PVPP, Povidone, a talc and a stearate. The thioaptamer may also include one or more inactives and may even be lyophilized. The thioaptamer may also be provided for resuspension or may be suspended for delivery intravenously, intraperitoneally, intramuscularly, subcutaneously, intracutaneously, alveolarly, sublingual or combinations thereof. The thioaptamer of may include thioate and sequence substitutions of the oligonucleotides identified by SEQ ID NOS.: 1, 2 and the combination thereof, e.g., the sequence of the formula:

5′ATGCTTCCACGAGCCTTTCGGGGTTGGTGTsACsAGTGGsATGGCTGCGsAGGCGGT sAGTCTsATTC 3′ (SEQ ID NO.:1), wherein s is a thioate substitution; and/or

3′TAsCGAsAsGGTGCTCGGAsAsAsGCCCCAsAsCCAsCAsTGTCAsCCTAsCCGACGCTC CGCCATCAGATAAG 5′ (SEQ ID NO.:2), wherein s is a thioate substitution.

The thioaptamer of may also be provided along with one or more pharmaceutically acceptable salts. According to one embodiment of the present invention, the partially-modified nucleotide aptamer (“thioaptamer”) may include one or more, but not all the backbone links as phosphoromonothioate or phosphorodithioate (“phosphorothioates”) and may be DNA or RNA. Examples of the modifications include: dATP(αS), dTTP(αS), dCTP(αS) and/or dGTP(αS), dATP(αS₂), dTTP(αS₂), dCTP(αS₂) and/or dGTP(αS₂), mixtures and combinations thereof in which a combination of the sequence and selected modifications bind specifically to RNase H. When in the form of RNA or DNA, the thioaptamer is modified accordingly in the backbone and the bases. In another embodiment of the present invention, no more than three adjacent phosphate sites of the modified nucleotide aptamer are replaced with phosphorothioate groups. In yet another embodiment of the present invention, at least a portion of non-adjacent dA, dC, dG, or dT phosphate sites of the modified nucleotide aptamer are replaced with phosphorothioate groups. In yet another embodiment of the present invention, all of the non-adjacent dA, dC, dG, or dT phosphate sites of the modified nucleotide aptamer are replaced with phosphorothioate groups. In yet another embodiment of the present invention, all of the non-adjacent dA, dC, dG, and dT phosphate sites of the modified nucleotide aptamer are replaced with phosphorothioate groups. In still another embodiment of the present invention, substantially all non-adjacent phosphate sites of the modified nucleotide aptamer are replaced with phosphorothioate groups.

### BRIEF DESCRIPTION OF THE DRAWINGS

For a more complete understanding of the features and advantages of the present invention, reference is now made to the detailed description of the invention along with the accompanying figures and in which:

FIG. 1 is an example of the two strands of an RNase H specific thio-aptamer (R12-2);

FIG. 2 shows a summary of examples of sequences of the selected thioaptamers (only the variable region is shown) that bind with specificity to RNase H;

FIG. 3 is a graph that shows the binding characteristics of one thioaptamer of the present invention, R12-2, to different proteins using EMSA (R12-2 shows binding to the RNase H domain of HIV RT protein (▪) but not to the E. coli RNase H (●). Initial library clone (★) did not show binding to the HIV RT, the intensity of the free-DNA band is plotted against the protein concentrations);

FIGS. 4A and 4B show EMSA binding of the lead thioaptamer R12-2 to HW RT (FIG. 4A, Gel electrophoresis mobility shift assay showing binding of R12-2 to the HIV RT. The arrows show the positions of upper and lower bands. Lane 1, no protein. Lanes 2-8 contain proteins in increasing concentrations (0.05, 0.1, 0.2, 0.4, 0.6, 0.8 and 1 μM, respectively); and FIG. 4B is a graph with a quantitative measurement of the intensity of the DNA band bound to the protein; intensity of the upper band is plotted against the HIV RT concentration in μM);

FIG. 5 is a graph of the effect of the selected thioaptamer R12-2 on RNase H activity of HIV RT (radioactivity retained in the filtrate, considered as a measure of RNase H activity, is plotted against the thioaptamer concentration);

FIGS. 6A and 6B are graphs that show that the thioaptamer R12-2 inhibits HIV-1 infection in U373-MAGI-CCR5 cells (FIG. 6A, cells infected 24 hrs after transfection with thioaptamer or controlled conditions were lysed after 48 hirs and analyzed for luciferase activity, as measured by relative light units (RLU). Controls included oligofectin (OF) transfection only, treatment with thioaptamers without OF, infection with no other treatment (SF 162 only) and treatment with AZT for 20 hrs prior to and 48 hrs after infection. FIG. 6B is a graph that also include a scrambled thioaptamer of the same size and base composition as R-12-2);

FIGS. 7A, 7B and 7C are graphs that show the dose response curves of thioaptamer R12-2 targeting RNase H domain of HIV-1 RT or XBY-S2 treatment targeting the human immunomodulatory transcription factor AP-1 on human U373-MAGI-CCR5 cells. U373-MAGI-CCR5 cells were transfected with various doses of thioaptamer 24 hrs prior to infection with HIV SF162, and were analyzed for luciferase activity (RLU) after 48 hrs. Results are presented as % of virus production compared with untreated controls. FIG. 7A is a graph of a dose curve of the response to thioaptamer R12-2; and FIG. 7B is a dose response curve of thioaptamer XBY-S2. C, Control studies performed in parallel with dose response studies;

FIG. 8 is a graph that shows the effect of thioaptamer R12-2 with increasing virus inocula when two-fold dilutions of SF162 supernatant were used to infect U373-MAGI-CCR5 cells 24 hrs after thioaptamer transfection or control condition, and infection was quantified in cell lysates after 48 hrs (RLU). Closed circles (●) represent untransfected cells, closed squares (▪) represent cells transfected with R12-2, and open triangles (Δ) represent control AZT (10 μM).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. The following abbreviations are used throughout: EMSA electrophoretic mobility shift assay; RNase, ribonuclease; HAART, highly active anti-retroviral therapy; HIV-1, human immunodeficiency virus type 1; HSQC, heteronuclear single quantum coherence; m.o.i., multiplicity of infection; NMR, nuclear magnetic resonance; NOESY, nuclear Overhauser enhancement spectroscopy; ODN, oligonucleotide agents; RT, reverse transcriptase.

As used herein, “synthesizing” of a random combinatorial library refers to chemical methods known in the art of generating a desired sequence of nucleotides including where the desired sequence is random. Typically in the art, such sequences are produced in automated DNA synthesizers programmed to the desired sequence. Such programming can include combinations of defined sequences and random nucleotides.

“Random combinatorial oligonucleotide library” is used to describe a large number of oligonucleotides of different sequence where the insertion of a given base at given place in the sequence is random. “PCR primer nucleotide sequence” refers to a defined sequence of nucleotides forming an oligonucleotide which is used to anneal to a homologous or closely related sequence in order form the double strand required to initiate elongation using a polymerase enzyme. “Amplifying” means duplicating a sequence one or more times. Relative to a library, amplifying refers to en masse duplication of at least a majority of individual members of the library.

As used herein, “thiophosphate” or “phosphorothioate” are used interchangeably to refer analogues of DNA or RNA having sulphur in place of one or more of the non bridging oxygens bound to the phosphorus. Monothiophosphates or phosphoromonothioates [αS] have only one sulfur and are thus chiral around the phosphorus center. Dithiophosphates are substituted at both oxygens and are thus achiral. Phosphoromonothioate nucleotides are commercially available or can be synthesized by several different methods known in the art. Chemistry for synthesis of the phosphorodithioates has been developed by one of the present inventors as set forth in U.S. Pat. No. 5,218,088, issued to Gorenstein, D. G. and Farschtschi, N., issued Jun. 8, 1993, for a Process for Preparing Dithiophosphate Oligonucleotide Analogs via Nucleoside Thiophosphoramidite Intermediates, relevant portions incorporated herein by reference.

When discussing changes to oligonucleotides, “modified” is used herein to describe oligonucleotides or libraries in which one or more of the four constituent nucleotide bases of an oligonucleotide are analogues or esters of nucleotides normally comprising DNA or RNA backbones and wherein such modification confers increased nuclease resistance. Thiophosphate nucleotides are an example of modified nucleotides. “Phosphodiester oligonucleotide” means a chemically normal (unmodified) RNA or DNA oligonucleotide. Amplifying “enzymatically” refers to duplication of the oligonucleotide using a nucleotide polymerase enzyme such as DNA or RNA polymerase. Where amplification employs repetitive cycles of duplication such as using the “polymerase chain reaction”, the polymerase may be, e.g., a heat stable polymerase, e.g., of Thermus aquaticus or other such polymerases, whether heat stable or not. When discussing the effect of an thioaptamer on RNase, the term “modified” is used to describe a change in the activity of the RNase domain, either alone or in conjunction with a reverse transcriptase or other enzymatic activity or related proteins that may upregulate or downregulate the activity of RNase activity, including, e.g., RNA degradation, DNA nicking, up or down regulation of gene expression by affecting gene degradation.

“Contacting” in the context of target selection means incubating an oligonucleotide library with target molecules. “Target molecule” means any molecule to which specific aptamer selection is desired. “Target protein” means any peptide or protein molecule to which a specific aptamer selection is desired. “Essentially homologous” means containing at least either the identified sequence or the identified sequence with one nucleotide substitution. “Isolating” in the context of target selection means separation of oligonucleotide/target complexes, preferably DNA/protein complexes, under conditions in which weak binding oligonucleotides are eliminated.

By “split synthesis” it is meant that each unique member of the combinatorial library is attached to a separate support bead on a two (or more) column DNA synthesizer, a different thiophosphoramidite or phosphoramidite is first added onto both identical supports (at the appropriate sequence position) on each column. After the normal cycle of oxidation (or sulfurization) and blocking (which introduces the phosphate, monothiophosphate or dithiophosphate linkage at this position), the support beads are removed from the columns, mixed together and the mixture reintroduced into both columns. Synthesis may proceed with further iterations of mixing or with distinct nucleotide addition.

Aptamers may be defined as nucleic acid molecules that have been selected from random or unmodified oligonucleotides (“ODN”) libraries by their ability to bind to specific targets or “ligands.” An iterative process of in vitro selection may be used to enrich the library for species with high affinity to the target. The iterative process involves repetitive cycles of incubation of the library with a desired target, separation of free oligonucleotides from those bound to the target and amplification of the bound ODN subset using the polymerase chain reaction (“PCR”). The penultimate result is a sub-population of sequences having high affinity for the target. The sub-population may then be subcloned to sample and preserve the selected DNA sequences. These “lead compounds” are studied in further detail to elucidate the mechanism of interaction with the target.

“Detectable labels” are compounds and/or elements that can be detected due to their specific functional properties and/or chemical characteristics, the use of which allows the agent to which they are attached to be detected, and/or further quantified if desired, such as, e.g., an enzyme, an antibody, a linker, a radioisotope, an electron dense particle, a magnetic particle and/or a chromophore or combinations thereof, e.g., fluorescence resonance energy transfer (FRET); There are many types of detectable labels, including fluorescent labels, which are easily handled, inexpensive and nontoxic.

The present inventors recognized that it is not possible to simply substitute thiophosphates in a sequence that was selected for binding with a normal phosphate ester backbone oligonucleotide. Simple substitution was not practicable because the thiophosphates can significantly decrease (or increase) the specificity and/or affinity of the selected ligand for the target. It was also recognized that thiosubstitution leads to a dramatic change in the structure of the aptamer and hence alters its overall binding affinity.

The present invention takes advantage of the “stickiness” of thio- and dithio-phosphate ODN agents to enhance the affinity and specificity to a target molecule. In a significant improvement over existing technology, the method of selection concurrently controls and optimizes the total number of thiolated phosphates to decrease non-specific binding to non-target proteins and to enhance only the specific favorable interactions with the target. The present invention permits control over phosphates that are to be thio-substituted in a specific DNA sequence, thereby permitting the selective development of aptamers that have the combined attributes of affinity, specificity and nuclease resistance.

In one embodiment of the present invention, a method of post-selection aptamer modification is provided in which the therapeutic potential of the aptamer is improved by selective substitution of modified nucleotides into the aptamer oligonucleotide sequence. An isolated and purified target binding aptamer is identified and the nucleotide base sequence determined. Modified achiral nucleotides are substituted for one or more selected nucleotides in the sequence. In one embodiment, the substitution is obtained by chemical synthesis using dithiophosphate nucleotides. The resulting aptamers have the same nucleotide base sequence as the original aptamer but, by virtue of the inclusion of modified nucleotides into selected locations in the sequences, improved nuclease resistance and affinity is obtained.

RNA and DNA oligonucleotides (ODNs) can act as “aptamers,” (i.e., as direct in vivo inhibitors selected from combinatorial libraries) for a number of proteins, including viral proteins such as HIV RT and transcription factors such as, e.g., human NF-κB, AP-1, NF IL-6 or other proteins involved in, e.g., transcription. Decoy ODNs were developed to inhibit expression from CRE and AP-1 directed transcription in vivo and inhibit growth of cancer cells in vitro and in vivo. Yet others have demonstrated the potential of using specific decoy and aptamer ODNs to bind to various proteins, serve as therapeutic or diagnostic reagents, and to dissect the specific role of particular transcription factors in regulating the expression of various genes. In contrast to antisense agents, duplex aptamers appear to exhibit few if any non-specific effects.

Among a large variety of modifications, S—ODN and S₂—ODN render the agents more nuclease resistant. The first antisense therapeutic drug uses a modified S—ODN. The S₂—ODNs also show significant promise, however, the effect of substitution of more nuclease-resistant thiophosphates cannot be predicted, since the sulfur substitution can lead to significantly decreased (or increased) binding to a specific protein as well as structural perturbations and thus it is not possible to predict the effect of backbone substitution on an aptamer selected combinatorially. Hence, the present inventors recognized that selection should be carried out simultaneously for both phosphate ester backbone substitution and base sequence.

Phosphorodithioate analogs have been synthesized to produce an important class of sulfur-containing oligonucleotides, the dithiophosphate S₂—ODNs. These dithioates include an internucleotide phosphodiester group with sulfur substituted for both non-linking phosphoryl oxygens, so they are both isosteric and isopolar with the normal phosphodiester link, and are also highly nuclease resistant. One group showed highly effective protection of the dithioate against degradation by endogenous nucleases after 58% backbone modification. Significantly, the S₂—ODNs, in contrast to the phosphoramidite-synthesized monothiophosphate (S—ODNs), are achiral about the dithiophosphate center, so problems associated with diastereomeric mixtures are completely avoided. The S₂—ODNs and the S—ODNs, are taken up efficiently by cells, especially if encapsulated in liposomes.

Thiophosphate aptamers are capable of specifically and non-specifically binding to proteins. Importantly, the present inventors have observed that sulfurization of the phosphoryl oxygens of oligonucleotides often leads to their enhanced binding to numerous proteins. The dithioate agents, for instance, appear to inhibit viral polymerases at much lower concentrations than do the monothiophosphates, which in turn are better than the normal phosphates, with K_{d}'s for single strand aptamers in the nM to sub-nM range for HIV-1 RT and NF-κB. For HIV-1 RT, dithioates bind 28-600 times more tightly than the normal aptamer oligonucleotide or the S-analogue. Sequence is also important, as demonstrated by the observation that a 14-nt dithioate based on the 3′ terminal end of human tRNA^{Lys} complementary to the HIV primer binding site is a more effective inhibitor (ID₅₀=4.3 nM) than simply dithioate dC₁₄ (ID₅₀=62 nM) by an order of magnitude.

Oligonucleotides with high monothio- or dithiophosphate backbone substitutions appear to be “stickier” towards proteins than normal phosphate esters, an effect often attributed to “non-specific interactions.” One explanation for the higher affinity of the thiosubstituted DNAs is the poor cation coordination of the polyanionic backbone sulfur, being a soft anion, does not coordinate as well to hard cations like Na⁺, unlike the hard phosphate oxyanion. The thiosubstituted phosphate esters then act as “bare” anions, and since energy is not required to strip the cations from the backbone, these agents appear to bind even more tightly to proteins.

As used herein, the terms “thio-modified aptamer,” “thioaptamer” and/or “partially thio-modified aptamer” are used interchangeably to describe oligonucleotides (ODNs) (or libraries of thioaptamers) in which one or more of the four constituent nucleotide bases of an oligonucleotide are analogues or esters of nucleotides that normally form the DNA or RNA backbones and wherein such modification confers increased nuclease resistance; and the DNA or RNA may be single or double stranded. For example, the modified nucleotide thioaptamer can include one or more monophosphorothioate or phosphordithioate linkages selected by incorporation of modified backbone phosphates through polymerases from wherein the group: dATP(αS), dTTP(αS), dCTP(αS), dGTP(αS), rUTP (αS), rATP(αS), rCTP(αS), rGTP(αS), dATP(αS₂), dTTP(αS₂), dCTP(αS₂), dGTP(αS₂), rATP(αS₂), rCTP(αS₂), rGTP(αS₂) and rUTP(αS₂) or modifications or mixtures thereof. Phosphoromonothioate or phosphorodithioate linkages may also be incorporated by chemical synthesis or by DNA or RNA synthesis by a polymerase, e.g., a DNA or an RNA polymerase or even a reverse transcriptase, or even thermostable or other mutant versions thereof. In another example, no more than three adjacent phosphate sites of the modified nucleotide aptamer are replaced with phosphorothioate groups. In yet another example, at least a portion of non-adjacent dA, dC, dG, dU or dT phosphate sites of the modified nucleotide aptamer are replaced with phosphorothioate groups. In another example of a thioaptamer, all of the non-adjacent dA, dC, dG, or dT phosphate sites of the modified nucleotide aptamer are replaced with phosphorothioate groups; all of the non-adjacent dA, dC, dG, and dT phosphate sites of the modified nucleotide aptamer are replaced with phosphorothioate groups; or substantially all non-adjacent phosphate sites of the modified nucleotide aptamer are replaced with phosphorothioate groups. In still another embodiment of the present invention, no more than three adjacent phosphate sites of the modified nucleotide aptamer are replaced with phosphorodithioate groups. The thioaptamers may be obtained by adding bases enzymatically using a mix of four nucleotides, wherein one or more of the nucleotides are a mix of unmodified and thiophosphate-modified nucleotides, to form a partially thiophosphate-modified thioaptamer library. In another example of “thioaptamers” these are made by adding bases to an oligonucleotide wherein a portion of the phosphate groups are thiophosphate-modified nucleotides, and where no more than three of the four different nucleotides are substituted on the 5′-phosphate positions by 5′-thiophosphates in each synthesized oligonucleotide are thiophosphate-modified nucleotides.

Thioaptamers and other nucleic acid analogs (e.g., peptide nucleic acids (PNAs), methylphosphonates, etc.) are emerging as important agents in therapeutics, drug discovery and diagnostics. Three key attributes define the unique ability of (thio)aptamers to perform their essential functions: (1) they target specific proteins in physiological pathways; (2) their sequence and structure is not intuitively obvious from canonical biologics and oftentimes can only be deduced by combinatorial selection against their targets; and (3) they bind their targets with higher affinities than do naturally occurring nucleic acid substrates. Importantly, the backbone modifications of thioaptamers and their nucleic acid backbone analogs enable aptamers to be introduced directly into living systems with in vivo lifetimes many times greater than unmodified nucleic acids, due to their inherent nuclease resistance of the modified aptamers. The inherent nuclease resistance is extraordinarily important for their efficacy in use.

In vitro combinatorial selection of thiophosphate aptamers may be used with the present invention. A recent advance in combinatorial chemistry has been the ability to construct and screen large random sequence nucleic acid libraries for affinity to proteins or other targets. The aptamer and/or thioaptamer nucleic acid libraries are usually selected by incubating the target (protein, nucleic acid or small molecule) with the library and then separating the non-binding species from the bound. The bound fractions may then be amplified using the polymerase chain reaction (PCR) and subsequently reincubated with the target in a second round of screening. These iterations are repeated until the library is enhanced for sequences with high affinity for the target. However, agents selected from combinatorial RNA and DNA libraries have previously always had normal phosphate ester backbones, and so would generally be unsuitable as drugs or diagnostics agents that are exposed to serum or cell supernatants because of their nuclease susceptibility. The effect of substitution of nuclease-resistant thiophosphates cannot be predicted, since the sulfur substitution can lead to significantly decreased (or increased) binding to a specific protein.

The present inventors have described the combinatorial selection of phosphorothioate oligonucleotide aptamers from random or high-sequence-diversity libraries, based on tight binding to the target (e.g. a protein or nucleic acid) of interest, U.S. patent application Ser. No. 10/120,815, relevant portions incorporated herein by reference. An in vitro selection approach for RNA thioaptamers has also been described Ellington and co-workers.

One approach used by the inventors is a hybrid monothiophosphate backbone. Competition assay for binding 42-mer aptamers (ODN) were conducted. In standard competitive binding assays, ³²P-IgkB promoter element ODN duplex was incubated with recombinant p50 or p65 and competitor oligonucleotide. The reactions were then run on a nondenaturing polyacrylamide gel, and the amount of radioactivity bound to protein and shifted in the gel was quantitated by direct counting.

In another example a combinatorial library was created by PCR, using an appropriate dNTP(αS) in the Taq polymerization step. A combinatorial thiophosphate duplex and single stranded (ss) libraries was screened successfully for binding to a number of different protein and nucleic acid targets, including: TGF-beta, NF-IL6, NF-κB, HIV reverse transcriptase, Venezuelan Equine Encephalitis nucleocapsid (using an RNA thioaptamer), HepC IRES nucleic acid, and others, including Interferon-gamma.

Briefly, a filter binding method was used that was modified to minimize non-specific binding of the S—ODNs to the nitrocellulose filters. A column method may also be used in which the target is covalently attached to a column support for separation as well. The duplex, ssDNA and/or ssRNA S—ODN's are eluted from the filter under high salt and protein denaturing conditions. Subsequent ethanol precipitation and for the duplex DNA S—ODNs, another Taq polymerase PCR thiophosphate amplification provided product pools for additional rounds of selection (for RNA thioaptamers RT and T7 polymerase were used). To increase the binding stringency of the remaining pool of S—ODNs in the library and select higher-affinity members, the KCl concentration was increased and the amount of protein in subsequent rounds was reduced as the iteration number increased. After cloning, the remaining members of the library were sequenced, which allowed for “thioselect”™ simultaneously for both higher affinity and more nuclease-resistant, “thioaptamer™” agents. For example, the thioselection method has been used to isolate a tight-binding thioaptamer for 7 of 7 targets tested.

### EXAMPLE 1

S—ODN, S₂—ODN and monothio-RNA Split and Pool Synthesis. A split and pool synthesis combinatorial chemistry method was developed for creating combinatorial S—ODN, S₂—ODN and monothio-RNA libraries (and readily extended to unmodified ODNs-whether single strand or duplex). In this procedure each unique member of the combinatorial library was attached to a separate support bead. Targets that bind tightly to only a few of the potentially millions of different support beads can be selected by binding the targets to the beads and then identifying which beads have bound target by staining and imaging techniques. The methodology of the present invention allowed the rapid screening and identification of thioaptamers that bind to proteins such as NF-κB using a novel PCR-based identification tag of the selected bead.

The dA, dG, dC and dT phosphoramidites were purchased from Applied Biosystems (Palo Alto, Calif.) or Glen Research (Sterling, Va.). The Beaucage reagent (³H-1,2-Benzodithiol-3-one 1,1-dioxide) was from Glen Research. The Taq polymerase kits were from Applied Biosystems. The TA Cloning kit was from Invitrogen. The Klenow DNA polymerase I was from Promega. Polystyrene beads (60-70 μm) with non-cleavable hexaethyleneglycol linkers with a loading of 36 μmol/g were from ChemGenes Corp (Ashland, MA). The Alexa Fluor 488 dye was from Molecular Probes, Inc (Eugene, Oreg.). The dA, dG, dC and dT thiophosphoramidites were synthesized as previous described (Yang, X-B., Fennewald, S., Luxon, B. A., Aronson, J., Herzog, N. and Gorenstein, D. G., “Aptamers containing thymidine 3′-O-phosphorodithioates: Synthesis and binding to Nuclear Factor-κB, J. Bioorganic and Medicinal Chemistry, 9, 3357-3362 (1999) and refs therein). The ODNs and S—ODNs used in the study were synthesized on a 1-μmol scale on an Expedite 8909 System (Applied Biosystems) DNA Synthesizer.

Combinatorial selection of the thioaptamers disclosed was employed to isolate double-stranded DNA thioaptamers that specifically target the RNase H protein. In one embodiment, the RNase H activity is part of a protein with additional enzymatic activity, e.g., a reverse transcriptase (RT) such as an HIV RT. Combinatorial selection of aptamers, and specifically, thioaptamers is advantageous in this respect, in that the selection isolates aptamers and thioaptamers that satisfy the structural requirement, unlike other methods of inhibitor selection such as the screening of small molecule libraries. The critical features of a thioaptamer selection process will be discussed hereinbelow.

The initial thioaptamer selection process of the present invention was designed to modify the backbone of double-stranded DNA aptamers, with phosphoromonothioate substitutions 5′ of both A and C nucleotides. Thioation provides enhanced nuclease resistance as well as increased affinity and specificity relative to unmodified phosphate aptamers. Sequence data on the clones isolated during the selection, the predicted secondary structure of the clones, preliminary binding data and predicted dimeric models of the clones based on this data are described below.

The enabling technology used in the selection process, combinatorial selection and isolation of phosphorothioate DNA aptamers (thio-PCR, isolation of single strand DNA), analysis of DNA aptamer sequences and of target proteins, and evaluation of binding affinities of the selected DNA aptamers, has been covered in a series of patent applications and an issued patent of the primary inventor (U.S. Pat. No. 6,423,493; U.S. Pat. No. 6,867,289, issued Mar. 15, 2005; U.S. patent application Ser. No. 10/214,417; U.S. patent application Ser. No. 10/272,509; U.S. Patent Application Ser. No. 60/472,890), relevant portions incorporated herein by reference).

Despite the key role played by the RNase H of Human Immunodeficiency Virus-Reverse Transcriptase (HIV-RT) in viral proliferation, only a few inhibitors of RNase H have been reported. Using in vitro combinatorial selection methods and the RNase H domain of the HIV-RT, the present inventors have selected double-stranded DNA thioaptamers (aptamers with selected thiophosphate backbone substitution) that inhibit RNase H activity and viral replication. The selected thioaptamer sequences exhibit a very high proportion of G residues. The consensus sequence for the selected thioaptamers show G clusters separated by single residues at the 5′ end of the sequence. Gel electrophoresis mobility shift assays and NMR studies show binding of the selected thioaptamer to the isolated RNase H domain, but did not bind to a structurally similar RNase H from E. coli. One examples of the thioaptamers selected herein, R12-2, shows specific binding to HIV-RT with a binding constant of 70 nm. Although not effective against the isolated RNase H domain, the thioaptamer inhibited the RNase H activity in the intact HIV-RT. In cell culture, transfection of thioaptamer R12-2 (0.5 mg/ml) markedly reduces viral production, and shows a dose response of inhibition with doses of R12-2 ranging from 0.03 mg/ml to 2.0 mg/ml (IC₅₀<100 nM). Inhibition was also seen across various ranges of virus inoculum, ranging from multiplicity of infection (m.o.i) of 0.0005 to 0.05, with reduction of virus production by more than 50% at high m.o.i. Suppression of virus was comparable to that seen with AZT at m.o.i.≦0.005.

Reverse transcription of viral genomic RNA into DNA, an essential step in the replication of retroviruses like the human immunodeficiency virus (HIV), is catalyzed by reverse transcriptase (RT). The bifunctional HIV-RT is a heterodimer with 66 and 51 kDa subunits (1). Both subunits contain a DNA polymerase domain. The 66 kDa domain, p66, contains an RNase H domain in addition to the DNA polymerase domain. The DNA polymerase uses both RNA and DNA templates to accomplish viral genomic replication. The RNase H catalyzes the cleavage of the viral RNA strand from the DNA:RNA hybrid duplex, thereby releasing the copy of the viral DNA to ultimately integrate into the host cell's genome.

HIV infection progresses to AIDS at variable rates in virtually every infected individual over a period of several years in the absence of therapy. The first class of antiretroviral drugs approved and used to treat HIV infection were the RT inhibitors. Although newer drugs have been approved for use that target HIV protease, and most recently the HIV envelope, RT inhibitors remain an important component of combination therapy known as highly-active anti-retroviral therapy (HAART). Most commonly used HAART regimens typically include two or three RT inhibitors. Use of HAART, rather than less intensive antiretroviral regimens, has dramatically reduced the rates of progression to AIDS and has improved survival.

Currently available RT inhibitors target the polymerase activity and are either nucleoside analog drugs that bind at the polymerase active site or non-nucleoside inhibitors that bind to a different region of the HIV-RT. Therapeutic benefits are markedly diminished by the emergence of drug resistant strains and by the potential toxicity of the approved antiretroviral drugs. Resistance to these drugs emerges rapidly because of the highly error-prone reverse transcriptase, as well as by the potential of recombination between different strains when cells are co-infected. Due to extensive cross-resistance within each drug class that reduces antiviral activity and clinical benefit of sequential HAART, there is an urgent need to develop new agents and treatment approaches to fight AIDS. This search for alternate targets and new agents has led to a new class of RT inhibitors known as Oligonucleotide (ODN) Reverse Transcriptase Inhibitors (ONRTIs). ODNs are emerging as potential therapeutic agents that can be attractive alternatives for the chemical drugs.

ODNs operate via different mechanisms, such as the sequence-specific translational arrest of mRNA expression (antisense), RNA inhibition using specific short double-stranded RNA (siRNA), or by binding to and inhibiting RT or other HIV proteins/cellular receptors (aptamers or decoys). The antisense strategy, proposed for the potential treatment of several diseases including AIDS and cancer, is based on the binding of the ODNs to the mRNA by complementary base-pairing. The decoy or sense strategy rests on the selective binding of the ODNs, called aptamers, to a nucleic acid binding protein. The decoy aptamer mimics the natural ligand of the protein and therefore competes with it for complex formation with the protein. This association traps the protein in a nonfunctional complex. An advantage of aptamer over the antisense technology is that the aptamer ODNs do not have to overcome the thermodynamic penalty required for the unfolding of their own and the target's secondary and tertiary structures. RNA and DNA aptamers targeting several HIV proteins have been reported. This include the inhibition of HIV-RT's polymerase and RNase H activities (Andreola, M-L., Pileur, F., Calmels, C., Ventura, M., Tarrago-Litvak, L., Toulme, J. J., and Litvak, S. (2001) Biochemistry, 40, 10087-10094. Pileur, F., Andreola, M., Dausse, E., Michel, J., Moreau, S., Yamada, H., Gaidamakov, S. A., Crouch, R. J., Toulme, J. J., and Cazenave, C. (2003) Nucleic Acids Res. 31, 5776-5788), HIV integrase function, and the capsid protein NCp7.

Backbone modifications in ODNs are essential to increase the resistance for digestion by cellular nucleases. Sulfur substitutions of phosphoric oxygens of the DNA backbone, as in monothio and dithio aptamers, often enhance their binding to proteins. The present inventions recognized that a distinct problem is found when thio-modifying ODNS, namely, the affinity of the ODNs due to sulfur substitutions. Complete substitution of the backbone leads to non-specific binding leading to significant loss in specificity of the ODN to the target protein. Therefore, the number (and position) of thio substitutions have to be optimized to decrease the non-specific binding to non-target proteins while enhance the binding to the target protein.

Despite the key role played by the RNase H domain of the HIV-RT in the viral proliferation, aptamers that could specifically target this domain have not yet been developed. Although aptamers inhibiting RNase H activity have been reported, they were generally selected against the entire HIV-RT. Hannoush R N, Min K L, Damha M J. “Diversity-oriented solid-phase synthesis and biological evaluation of oligonucleotide hairpins as HIV-1 RT RNase H inhibitors” Nucleic Acids Res. 2004 November 29;32(21):6164-75 (2004). Unlike most other approaches, the present inventors used the isolated RNase H domain of the HIV-RT in the selection process to select RNase H-specific thioaptamers that bind specifically to the RNase H domain of the HIV-RT. In-vitro combinatorial selection of monothio-phosphate modified DNA thioaptamers was used to isolate, characterize and purify thioaptamers that bind selectively to the RNase H domain of HIV-RT, that inhibits RNase H activity, and that exhibit antiretroviral activity.

Materials. NTPs, and HIV-RT were purchased from Amersham Bioscience (Piscataway, N.J.). The initial DNA library and PCR primers were chemically synthesized by Midland Certified Reagents, (Midland, Tex.). ³H-UTP was purchased from NEN (Boston, Mass.). Chirally pure Sₚ isomer of dATP (αS) was obtained from Biolog Life Science Institute (Bremen, Germany). TOPO TA cloning kits were from Invitrogen (Carlsbad, Calif.), and the plasmid isolation kits were from Qiagen (Foster City, Calif.), AmpliTaq DNA polymerase and other PCR consumables were purchased from Applied Biosystems (Foster City, Calif.). Transfection agent Oligofectamine was purchased from Invitrogen.

Preparation of HIV-1 RNase H. RNase H was expressed in E. coli and purified as described previously (Becerra, S. P., Clore, G. M., Gronenborn, A. M., Karlstrom, A. R., Stahl, S. J., Wilson, S. H. and Wingfield, P. T., (1990) FEBS Letters 270, 76-80). A new T7 RNA polymerase/IPTG-inducible plasmid was developed that encodes the 15 kDa RNase H domain of HIV-RT. Both unlabelled and uniformly ¹⁵N labeled protein were purified to homogeneity after the over expression in minimal media or minimal media containing the ¹⁵NH₄Cl. This vector yields excellent quantities of pure RNase H (ca. 20 mg/L in an E. coli expression system). Expression tests in rich, minimal and ¹⁵N minimal media confirmed the presence of a 15 kDa fragment (by SDS PAGE). The authenticity of the protein was confirmed by N-terminal sequencing and NMR.

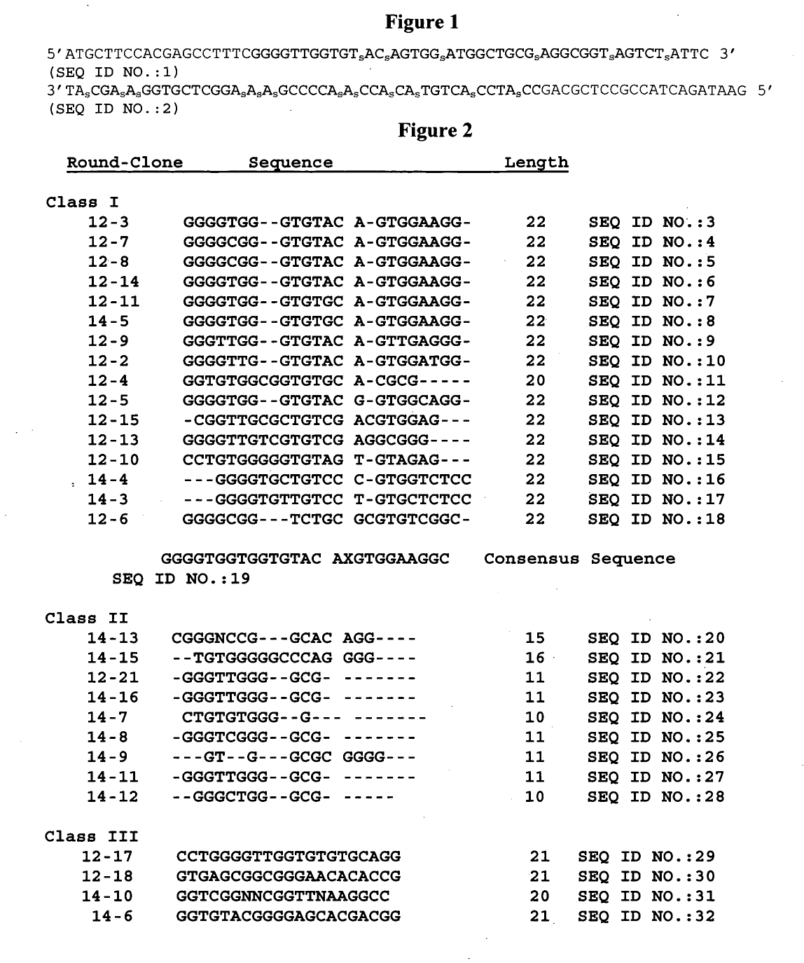

Synthesis of DNA libraries. The chemically synthesized random combinatorial library is a 62 nucleotide long single strand DNA containing a 22 nucleotide random region flanked by 19 and 21 nucleotide PCR primer regions (FIG. 1). The library was annealed with the reverse primer and subjected to Klenow reaction for 5 hrs at 37° C., followed by PCR amplification using AmpliTaq DNA polymerase and a mixture of dATP(αS), dTTP, dCTP, and dGTP to give the thioaptamer substituted library in which all the 5′ dA's were substituted with monothiophosphates. Reaction conditions for the PCR-amplifications were: oligonucleotides library (30 μM), dATP(αS) (1.6 mM), mixture of dTTP, dCTP, and dGTP (0.8 mM each), MgCl₂ (1 mM), primers (400 nM each) and AmpliTaq DNA polymerase (1U) in a total volume of 100 μl. PCR is run for 35-40 cycles of 94° C./2 min, 55° C./2 min and 72° C./2 min. The resulting 62-mer library contained monothiophosphate substitution at 5′ to every dA residue, in Sₚ configuration with the exception of the primer region on the non-template strand. Standard phosphoryl PCR amplification was carried out with a mixture of dNTP (0.8 mM each) along with the other reagents for 20-25 cycles of 94° C./1 min, 45° C./1 min and 72° C./1 min. For the EMSA, 5′-fluorescein labeled thioaptamers were synthesized using a PCR primer labeled with fluorescein at the 5′-end.

Combinatorial Selection of Thioaptamers. Purified RNase H protein was incubated with the pre-filtered (to exclude the thioaptamer sequences that could bind to nitrocellulose filters), PCR-amplified random monothio DNA library in a binding buffer containing 50 mM Tris-HCl, MgCl₂ (1.25 mM), KCl (25-400 mM) and DTT (10 mM) in a total volume of 50 μl for 1 hr at ambient temperature, followed by filtration with nitrocellulose filters. The filters were washed 4 times with the binding buffer (1 ml) to remove the unbound and weakly bound DNA. After the washes, thioaptamers bound to the protein in the DNA-protein complexes would be retained on the filter. A 0.5 ml solution of 8M urea and 2M KCl was then added to elute the thioaptamers bound to the protein. A negative control experiment without the protein was performed simultaneously to monitor any nonspecific binding of the thiophosphate library to the filter. The eluted DNA was extracted and PCR amplified to be taken to the next round of selection. The amplified DNA was analyzed by 15% non-denaturing polyacrylamide gel electrophoresis. The stringency of selection was tightened at each selection round by decreasing the amount of protein and gradually increasing the salt concentration in the binding buffer (from 25 mM to 400 mM). The initial library and the sequences obtained after rounds 5, 8, 12 and 14, were determined. A chemically synthesized double strand 14-mer with 6 dithiophosphate substitutions (XBY-S2) shown to target AP-1 was used as a control in virus inhibition studies (Yang, X., and Gorenstein, D. G., (2004) Current Drug Targets. 5, 705-715).

Electrophoresis Mobility Shift Assay (EMSA). One of the selected thioaptamers (R12-2) was used to test the specific binding to RNase H. R12-2 was labeled with fluorescein at the 5′-end by enzymatic synthesis and was incubated with increasing concentrations of HIV-RT RNase H in Tris-HCl buffer (50 mM. pH 8), MgCl₂ (10 mM) and DTT (40 mM) at 37° C. for 30 minutes. The reaction mixture was loaded on an 8% native polyacrylamide gel, electrophoresed and analyzed on the Flor Chem 8800 imager (Alpha Innotech, CA). Similar experiment was performed with the E.coli RNase H, to test whether R12-2 binds to the RNase H from E.coli.

HIV-RT RNase H activity assay. A DNA:RNA heteroduplex containing radiolabeled RNA was prepared as a substrate for the RNase H. A tritium-labeled RNA molecule, the stem loop 2 of the HIV psi-RNA (25), was made by in-vitro transcription using T7 RNA polymerase with a mixture of ATP, CTP, GTP and ³H-UTP. Transcribed RNA was purified by centrifugation using Microcon YM-3 filters, to remove NTPs and other short RNA sequences. The purified RNA was annealed with the complementary DNA strand to give the DNA:RNA hybrid where the RNA strand is ³H-labeled. HIV-RT (0.35 μM) was incubated with R12-2 thioaptamer (0.5-2 μM) in a buffer containing Tris-HCl (50 mM, pH 8.0), MgCl₂ (6 mM), DTT (10 mM) and KCl (80 mM) for 30 min at 37° C. To this mixture, labeled DNA-RNA hybrid (40 000 cpm) was added to a final volume of 50 μl and incubated at 37° C. for 20 min. The reaction was quenched by addition of 150 μl of 10% trichloroacetic acid and the mixture was placed on ice for 10 min. The reaction mixture was filtered, washed and the amount of radioactivity in the filtrate was measured using a scintillation counter (Beckman Coulter, CA). The amount of radioactivity in the filtrate is regarded as proportional to RNase H activity. A control experiment was performed without any HIV RT, and 100% of RNase H activity is assumed in the absence of any R12-2 thioaptamer.

NMR Spectroscopy. Uniformly ¹⁵N labeled RNase H and chemically synthesized R12-2 were used to monitor the binding of the R12-2 thioaptamer to the isolated domain of RNase H. The oligonucleotide concentrations were calculated from the absorbance value and using the molar absorption coefficients calculated using the nearest neighbor parameters. ¹⁵N-HSQC spectra (26) of the isolated RNase H in the presence of 25 mM MgCl₂ were acquired on Varian UnityPlus 750 MHz instrument equipped with pulse field gradients and using triple resonance probes. One dimensional proton NMR and 2D NOESY spectra (250 ms mixing time) of R12-2 thioaptamer in 95% H₂O/5% D₂O were collected at 750 MHz. The signals from the non-exchangeable imino protons of R12-2, that are very sensitive to changes in the DNA conformation, were monitored during the titration of RNase H to the R12-2.

Viruses and cells. HIV-1 SF162-R5 is a primary isolate that uses CCR5 as a coreceptor (R5), and was obtained from the NIH AIDS Research and Reference Reagent Program. U373-MAGI-CCR5 cells have modifications of the U373 astrocytoma adherent cells that allow use for HIV transfection and infection studies. U373-MAGI-CCR5 cells express β-gal under the control of HIV LTR, which is trans-activated by the HIV Tat protein in relation to the level of virus replication. In addition, these cells express CD4 and the human chemokine receptor CCR5 on its surface, which allow infection by primary HIV strains. U373 cells are propagated in 90% DMEM, 10% fetal bovine serum, 0.2 mg/ml G418, 0.1 mg/ml hydromycin B, and 1.0 μg/ml puromycin. For transfection and infection studies, U373 cells were maintained in 90% DMEM, 10% fetal bovine serum, and 1% penicillin/streptomycin. Thioaptamer RNase H-specific R12-2, control AP-1-specific or scrambled ODNs were transfected into the cells 24 hr prior to infection using Oligofectamine liposomes, according to the manufacturer's instructions (Invitrogen).

Infection of ODN-transfected U373-MAGI-CCR5 cells. Transfected cells and control cells were infected with HIV at m.o.i. of 0.003 (which consistently gives high levels of viral production) for 2 hr. Equal volumes of medium were then added and infection was continued for 48 hr. HIV replication was quantitated in cell lysates by measurement of β-gal activity, determined by luminometry using the β-Glo kit (Promega), according to the manufacturer's guidelines. β-gal activity is expressed as relative light units (RLU). To determine HIV TCID₅₀ in U373-MAGI-CCR5 cells, serial dilutions (10×) of SF162-R5 virus supernatant were innoculated onto a 24-well plate containing 3×10⁴ U373 cells for 2 hr, then equal volumes of complete medium were added, and infection was continued for 48 hr. Cells then were lysed and assayed for HIV-directed β-gal expression, and TCID₅₀ (50% tissue culture infective dose) was calculated from quadruplicates. The p24 antigen capture assay (Beckman-Coulter) was used to determine virus production in some studies, measured by p24 antigen concentration in the culture supernatants harvested at day 7 post-infection.

PCR Amplification of Thioaptamers. For the amplification of DNA sequences with monothio phosphate substitutions, α-S dATPs was used in the PCR reaction mixture (thio-PCR) along with dGTP, dTTP and dCTP. Only the Sₚ isomer of the αS dNTPs is used as substrate by the AmpiliTaq DNA Polymerase enzyme to yield the pure Rₚ stereoisomer (27). Yields from the thio-PCR are 20-35% lower compared to yields obtained using all four normal dNTPs. To compensate for the loss of yield in the thio-PCR, reaction conditions were changed by doubling the concentrations of α-S dATP, increasing the annealing temperature by about 10° C. and increasing the number of PCR cycles.

Sequences of Selected Thioaptamers. After 12 rounds of selection the monothio library converged to a few predominant sequences starting from a library of 10¹⁴ different sequences. FIG. 2 is a summary of the variable regions of the thioaptamers selected after the 12^{th} and 14^{th} rounds. On the basis of primary sequence alignments done by the ClustalW program (28), the selected sequences are grouped into three classes. Sequences in class I showed very high degrees of alignment, and those that do not align well were listed under class II. The third class contained aptamers with truncated variable regions. These thioaptamers had 10-15 base pairs in the variable region as opposed to 22 base-pairs in the original library. It is possible that the shorter thioaptamers could have been formed by inefficient or incomplete amplification of the library during the PCR cycles. However, sequences in all three classes showed similar sequence motifs. The sequences of the selected thioaptamers show G-rich sequence at their 5′ ends. These G rich sequences, with a minimum of four interspersed GG nucleotides, are shown to have the potential to form G-quartet structures (29, 30). Recent reports of single strand DNA aptamers selected against HIV-RT, HIV-1 integrase, and human RNase H also show G-rich sequences that could form G-quartet structures (14, 16, 31).

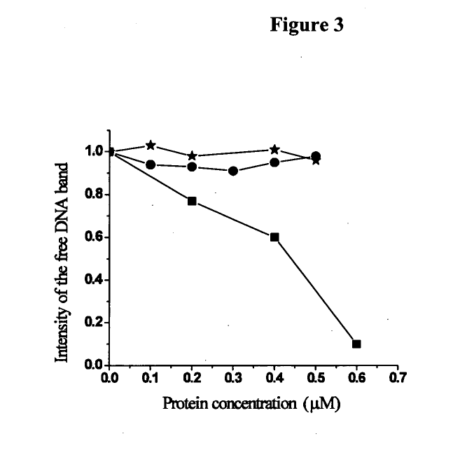

EMSAs show binding of the selected thioaptamers to RNase H of HIV-RT. As shown in FIG. 3 using EMSA, the R12-2 thioaptamer demonstrated specific binding to the isolated domain of the RNase H. In these gel electrophoresis studies, two bands are visible. The lower band corresponds to the free oligonucleotide and the shifted upper band corresponds to the oligonucleotide bound to the protein. The intensity of the free DNA band (lower band) decreased with increase in RNase H concentration, showing the increased binding of R12-2 with the increase in protein concentration. Under identical conditions, however, the R12-2 did not show any significant binding to the RNase H of E. coli, a structurally similar protein to the RNase H of HIV-RT. Similarly, the initial library did not show any binding to the RNase H of HIV-RT.

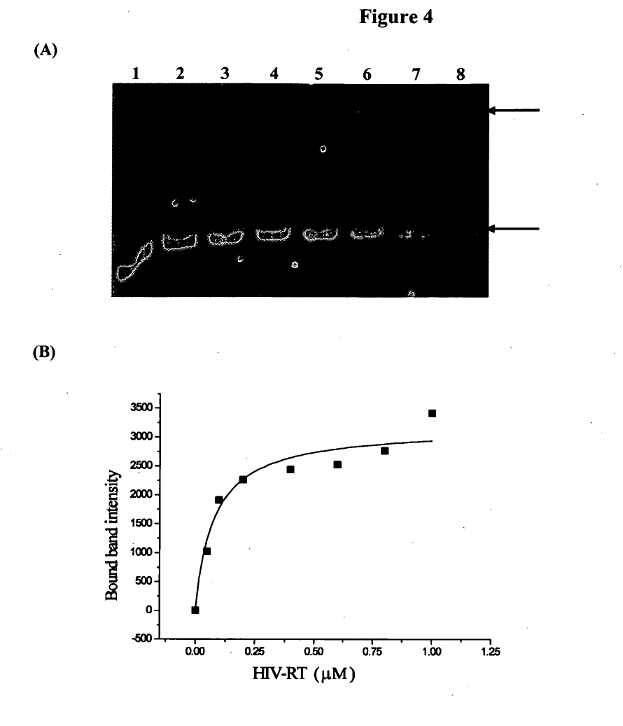

Next, the binding of R12-2 to the intact HIV-RT by EMSA methods was also determined. In FIG. 4a, the protein in the band was detected by staining the gel with Coomassie blue. With the increase in protein concentrations, the intensity of the protein-bound DNA band (upper band) increases while the intensity of the free DNA band (lower band) decreases. This observation shows that the R12-2 is binding to the HIV-RT. FIG. 4b shows the binding of thioaptamer R12-2 to the HIV-RT is quantitatively shown in the plot of bound band intensity vs. protein concentration. The value for the dissociation constant K_{d} determined from the plot, using non linear regression analysis on the Hill Plot method using the Origin software, is 70 nM.

NMR spectroscopy showed the interaction between R12-2 and the isolated domain of RNase H HIV-RT. Since the binding of R12-2 to the isolated domain of RNase H was not observed by electrophoresis mobility shift assay, NMR spectroscopic was used to demonstrate the binding of the R12-2 to the isolated RNase H domain. Imino proton signals of R12-2, the most sensitive indicators of the structural perturbations in DNA conformation, showed broadening with the addition of RNase H, indicating the binding of R12-2 to the protein. The linewidths of the imino proton signals increased with the increase in protein concentration and eventually disappeared.

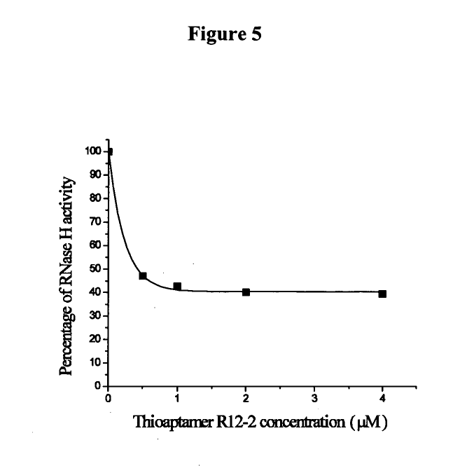

R12-2 shows inhibition of RNase H activity. The ability of the thioaptamer R12-2 to inhibit RNase H activity was tested using a radio-labeled substrate of a DNA:RNA hybrid duplex, as described in Methods. FIG. 5 shows the inhibition of RNase H activity using R12-2. The intact HIV-RT was used instead of the isolated RNase H domain. Although the RNase H domain can be expressed and isolated as a stable protein, it is shown to be inactive or very weakly active. The assay was based on the ability of the RNase H domain in HIV-RT to cleave the RNA strand in the RNA:DNA duplex.

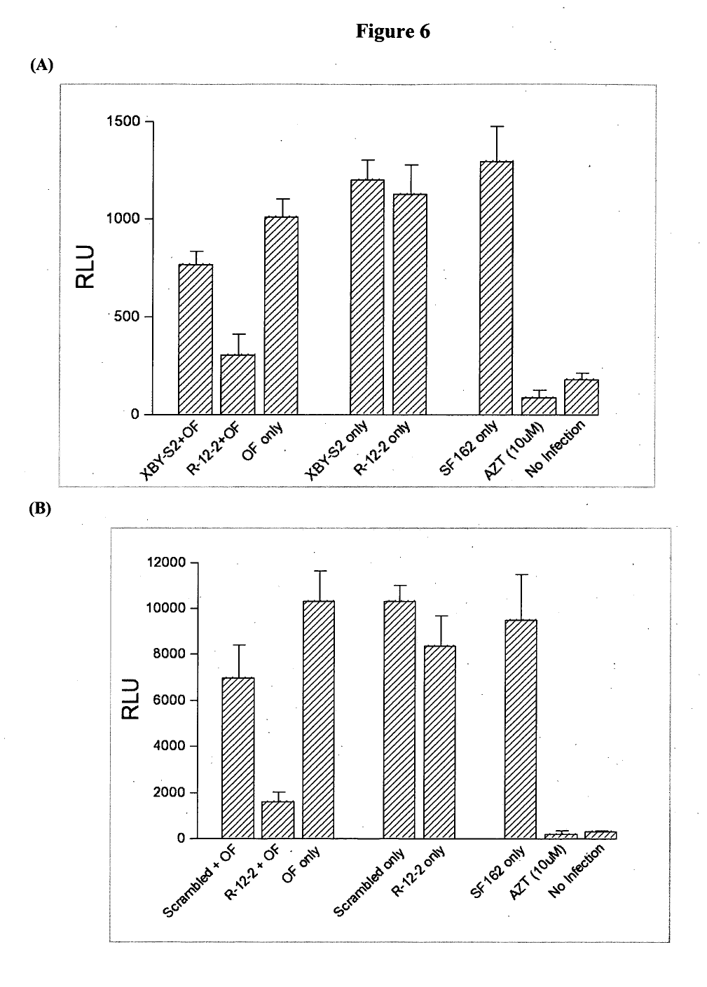

R12-2 thioaptamer inhibits HIV replication in vitro. Incubation of U373-MAGI-CCR5 cells, transfected with Oligofectamine (OF)-thioaptamer R12-2, 24 h prior to infection, with a prototypic primary HIV strain, HIV-SF162 (R5; at m.o.i. of 0.003). FIGS. 6A and 6B, show that R12-2 demonstrated greater than 75% reduction of infection, as compared with untransfected cells (exposed to R12-2 or XBY-S2 in absence of OF) or cells treated with OF reagent only. Chemically synthesized thioaptamer XBY-S2 (specific for human AP-1), showed only modest effects on HIV infection (FIG. 6A). In addition, a random thioaptamer of the same length and nucleoside composition as thioaptamer R12-2 showed no significant effect on HIV infection (FIG. 6B).

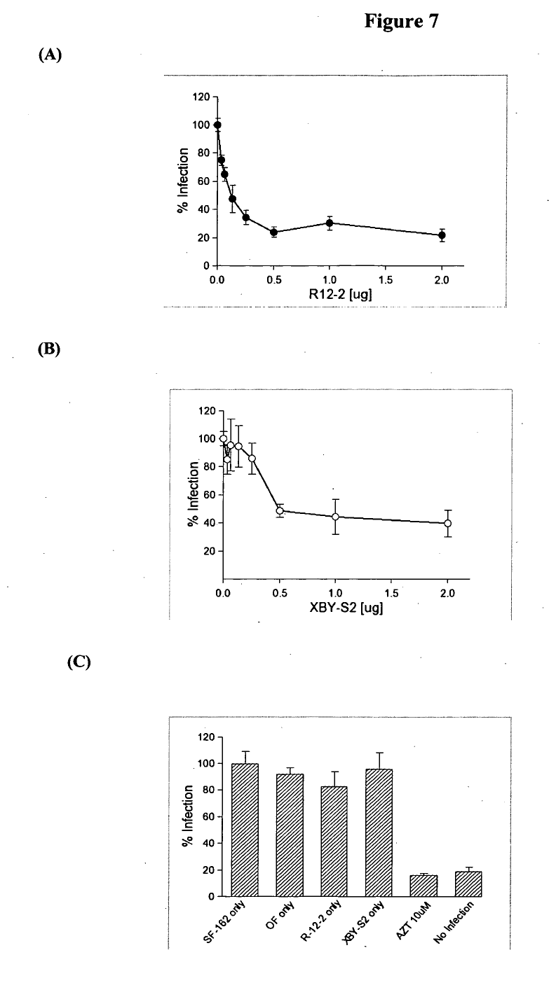

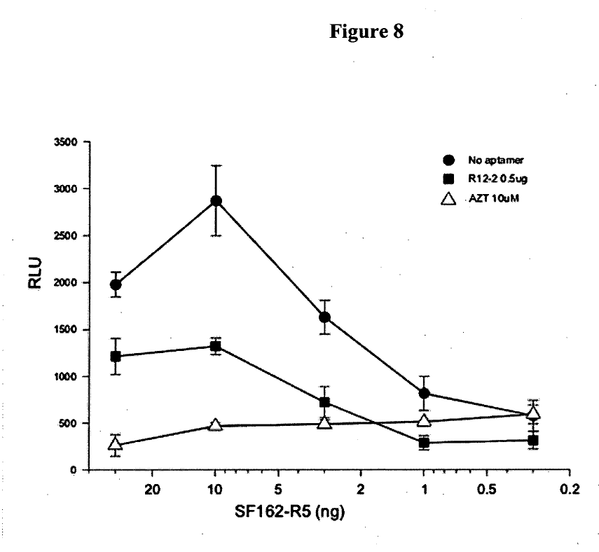

FIG. 7 shows that R12-2 demonstrated a dose-dependent inhibitory response with doses of thioaptamer R12-2 ranging from 0.03 to 2.0 μg/ml (IC₅₀<100 nM) to the same low level as 10 μM AZT. Transfection of thioaptamer R12-2 (0.05 μg/ml) results in inhibition of viral replication across a broad range of SF162 (R5) HIV-1 inocula ranging from an m.o.i. of 0.05 (30 ng) to 0.0005 (0.3 ng) as shown in FIG. 8. R12-2 shows significant inhibition of the virus at high m.o.i., and viral replication is comparable to that seen with AZT at m.o.i.≦0.005. (0.3 ng p24).

ODNs are emerging as promising alternative therapeutic agents to the currently used anti-AIDS drugs. However, to increase the resistance to digestion by cellular enzymes, modified ODNs have to be developed. Modifications in the phosphate backbone will significantly affect their binding to target proteins, since most of the direct contacts between the DNA-binding proteins and their binding sites in DNA are to the phosphate groups (34, 35). Sulfur substitutions of the phosphoric oxygens of ODNs often lead to their enhanced binding to numerous proteins (21, 22). However, complete substitution of phosphoryl oxygens with sulfur appears to make thioaptamers too “sticky” such that they lose their ability to discriminate between target and non-target proteins. Therefore, the total number and positions of thio-substituted phosphates have to be optimized using either rational design principles or by combinatorial techniques to decrease non-specific binding to non-target proteins, and enhance only the specific favorable interactions with RNase H. By using only the dATP (αS) during PCR amplification cycles, thioaptamers were produced that have thio-substitutions only at selected positions, 5′ to the A residues. Combinatorial selection methods that allow screening of large number of random sequence nucleic acid libraries for affinity to proteins facilitate the selection of ODNs that would have the optimal number of thio-phosphate substitutions. In one example, thioaptamer R12-2; demonstrated selective binding to the RNase H of HIV RT. This thioaptamer did not bind to the E. coli RNase H, even though these two proteins are structurally similar. By using combinatorial synthesis and selection methods and the selective sulfur substitution, a DNA thioaptamer was selected and further characterized with tight, specific binding to the RNase H of HIV-RT, inhibits the RNase H activity in-vitro and inhibits HIV replication in cell cultures.

Phosphorothioate modifications show increased resistance to digestion by cellular nucleases and often higher affinity for binding to proteins. They can also be readily synthesized in high yields and enhance favorable pharmacokinetic properties (36). Various pharmacokinetic studies in animals have shown that phosphorothioate analogs are rapidly dispersed to various tissues and cleared through the kidney within acceptable time periods (37).

Although the RNase H domain of the HIV-RT can be expressed and isolated as a stable protein, the isolated domain is either catalytically inactive or very weakly active. The loss of catalytic activity of the isolated domain is not likely due to structural differences between the two forms, since the structure. of the RNase H domain in isolation and in the intact RT are very similar (38, 39). The basis for the inactivity of the isolated domain is attributed mainly to the increased dynamics. Recent studies on the backbone dynamics using NMR of the isolated RNase H domain indicate that the intramolecular dynamic behavior of the isolated domain is severe resulting in the loss of catalytic activity of RNase H in isolation (40, 41). By only using the RNase H domain in the iterative selection rounds, the thioaptamers selected using the methods of the present invention specifically bind to the RNase H domain of RT. However, the R12-2 thioaptamer is a 62-mer duplex and other regions of the duplex very likely extend into the polymerase site of the RT, as observed in various duplex DNA-RT x-ray crystal structures and models (42, 43). The selected thioaptamer, R12-2, is shown to inhibit the function of the RNase H in the intact HIV-RT. Since the structures of the RNase H domain is similar in isolation and in the intact HIV-RT, selecting thioaptamers against the isolated domain that could inhibit the function of the enzyme in intact HIV-RT would be a better choice than selecting aptamers against the intact HIV-RT.

Inhibition was seen across a broad range of HIV inocula, and was comparable to AZT at low m.o.i. The inhibition seen at high m.o.i is noteworthy, since transfection efficiency in some studies is only 50%. Thus, compound activity may be underestimated when compared with drugs, such as AZT, which are taken up by essentially every viable cell in the culture.

As the HIV epidemic continues to mature through treatment error, emergence of resistance to the initial classes of antiretroviral drugs which includes, the HIV RT inhibitors, there will be a need to develop treatments directed to new targets. The various inhibitors of HIV entry, targeting attachment, co-receptor binding and fusion, will have a substantial impact on the ability to suppress viremia as they are developed for clinical use, by extending treatment options, but additional new classes of drug will inevitably be needed. Combinatorial selection of thioaptamers for binding to novel targets may allow development of therapeutic agents directed to diverse viral functions.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defmed by the appended claims.

### REFERENCES

1. Schatz, O., Mous, J., & Le Grice, S. F. J. (1990) EMBO J. 9, 1171-1176.
2. Palella, F. J Jr, Delaney, K. M., Moorman, A. C., Loveless, M. O., Fuhrer, J., Satten, G. A., Aschman, D. J., and Holmberg, S. D., (1998) N. Engl J. Med. 338, 853-860.
3. Yeni, P. G., Hammer, S. M., Hirsch, M. S., Saag, M. S., Schechter, M., Carpenter, C. C., Fisch, 1 M. A., Gatell, J. M., Gazzard, B. G., Jacobsen, D. M., Katzenstein, D. A., Montaner, J. S., Richman, D. D., Schooley, R. T., Thompson, M. A., Vella, S., Volberding, P. A., (2004) JAMA. 292, 251-265.
4. Hirsch, M. S., Brun-Vezinet, F., Clotet, B., Conway, B., Kuritzkes, D. R., D'Aquila, R. T., Demeter, L. M., Hammer, S. M., Johnson, V. A., Loveday, C., Mellors, J. W., Jacobsen, D. M., Richman, D. D., (2003) Clinical Infectious Diseases. 37, 113-128.
5. Ferguson, M. R., Rojo, D. R., von Lindern, J. J., and O'Brien, W. A. (2002) Clin. Lab. Med., 3, 611-635.
6. Majumdar, C., Stein, C. A., Cohen, J. S., Broder, S., and Wilson, S. H. (1989) Biochemistry, 28, 1340-1346.
7. Tuerk, C., MacDougal, S., and Gold, L., (1992) Proc. Nat!. Acad. Sci USA, 89, 6988-6992.
8. Stein, C. A., and Cheng, Y. C. (1993) Science, 261, 1004-1012.
9. (1996) Morgan, R. A., and Walker, R., (1996) Hum Gene Ther., 7, 1281-306.
10. Ellington, A. D., and Szosytak, J. W., (1990), Nature, 346, 818-822.
11. Gold, L., Polisky, B., Uhlenbeck, O. C., and Yarus, M. (1995) Annu. Rev. Biochem. 64, 763-797.
12. King D. J., Bassette, S. E., Li, X., Fennewalk, S. A., Herzog, N. K., Luxon, B. A., Shope, R., and Gorenstein, D. G. (2002) Biochemistry, 41, 9696-9706.
13. Agarwal, S. (1991). Antisense oligonucleotides: possible approaches for the chemotherapy of AIDS, p 143-159. In E. Wickstrom (ed), Prospects for antisense nucleic acid therapy of cancer and AIDS, Liss, New York, N.Y.
14. Schneider, D. J., Feigon, J., Hostomsky, Z. and Gold, L. (1995) Biochemistry, 34, 9599-9610.
15. Nickens, D. G., Patterson, J. T. and Burke, D. H. (2003) RNA, 9, 1029-1033.
16. Andreola, M-L., Pileur, F., Calmels, C., Ventura, M., Tarrago-Litvak, L., Toulme, J. J., and Litvak, S. (2001) Biochemistry, 40, 10087-10094.
17. Pileur, F., Andreola, M., Dausse, E., Michel, J., Moreau, S., Yamada, H., Gaidamakov, S. A., Crouch, R. J., Toulme, J. J., and Cazenave, C. (2003) Nucleic Acids Res. 31, 5776-5788.
18. de Solutrait, V. R., Lozach, P., Altmeyer, R., Tarrago-Litvak, L., Litvak, S., and Andreola (2002) J. Mol. Biol., 324, 195-203.
19. Paoletti A C, Shubsda M F, Hudson B S, Borer P N., (2002) Biochemistry 41, 423-428.
20. Shubsda M F, Paoletti A C, Hudson B S, Borer P N. (2002) Biochemistry 41, 5276-5282.
21. Milligan, J. F., and Uhlenbeck, O. C. (1989) Biochemistry, 28, 2849-2855.
22. Marshall, W. S., and Caruthers, M. H. (1993) Science, 259, 1564-1570.
23. Becerra, S. P., Clore, G. M., Gronenborn, A. M., Karlstrom, A. R., Stahl, S. J., Wilson, S. H. and Wingfield, P. T., (1990) FEBS Letters 270, 76-80.
24. Yang, X., and Gorenstein, D. G., (2004) Current Drug Targets. 5, 705-715
25. Amarasinghe, G. K., De Guzman, R. N., Turner, R. B., Chancellor, K. J., Wu, Z. R., Summers, M. F., (2000) J Mol Biol. 301, 491-511.
26. Bax, A., and Pochapsky, S. S. (1992) J. Magn. Reson. 99, 638-643.
27. Eckstein, F., and Thomson, J. B. (1995) Methods Enzymology, 262, 189-202.
28. Clustal W Program: Thompson, J. D., Higgins, D. G., Gibson, T. J., Nucleic Acids Res.; 22(22):4673-80.
29. Williamson, J. R., Raguraman, M. K., and Cech, T. R. (1989) Cell, 59, 871-880.
30. Smith, F. W., and Feigon, J. (1992) Nature, 356, 164-168.
31. Bianchi, A., and de Lange, T., (1999) J Biol Chem. 274, 21223-21227
32. Evans, D. B., Brawn, K., Deibel, M. R., Jr., Tarpley, W. G., and Sharma, S. K. (1991) J. Biol. Chem. 266, 20583-20585.
33. Hostomsky, Z. H., Z., Hudson, G. O., Moomaw, E. W., and Nodes, B. R. (1991) Proc. Natl. Acad. Sci. U.S.A. 88, 1148-1152.
34. Botuyan, M. V., Keire, D. A., Kroen, C., Gorenstein, D. G. (1993) Biochemistry, 32, 6863-6874.
35. Cho, Y., Zhu, F. C., Luxon, B. A., Gorenstein, D. G., (1993) J. Biomol Struct Dyn, 11, 685-702.
36. Tonkinson, J. L., Guvakova, M., Khaled, Z, Lee J., Yakubov, L, Marshall, W. S, Caruthers, M. H, Stein, C. A. (1994) Antisense Res Dev. 4, 269-78.
37. GalloBraz, M., et al, (2003) J. Med Biol Res. 36, 143-151.
38. Davies, J. F., II, Hostomska, Z., Hostomsky, Z., Jordan, S. R., and Matthews, D. A. (1991) Science 252, 88-95.
39. Chattopadhyay, D., Finzel, B. C., Munson, S. H., Evans, D. B., Sharma, S. K., Strakalaitis, N. A., Brunner, D. P., Eckenrode, F. M., Dauter, Z., Betzel, C., and Einspahr, H. M. (1993) Acta Crystallogr., Sect. D 49, 423-427.
40. Pari, K., Mueller, G. A., DeRose, E. F., Kirby, T. W., and London, R. E. (2003) Biochemistry, 42, 639-650
41. Mueller, G. A., Pari, K., DeRose, E. F., Kirby, T. W., and London, R. E., (2004) Biochemistry, 43, 9332-9342.
42. Jacobo_Molina, A., , Ding, J., Nanni, R. G., Clark, A. D., Lu, X., Tantillo, C., Williams, R. L., Kamer, G., Ferris, A. L., Clark, P., Hizi, A., Hughes, S. H., and Arnold, E. (1993) Proc Natl Acad Sci U S A. 90, 6320-6324.
43. Bebenek, K., Berad, W. A., Darden, T. A., Prasad, R., Luxon, B. A., Gorenstein, D. G., Wilson, S. M., and Kunkel, T. A. (1997) Nat. Str. Biol. 4, 194-197.
   Patents
U.S. Pat. No. 6,447,769, Compositions and methods for enhanced tumor cell immunity in vivo, H. Fakhrai, et al., issued Sep. 10, 2002.
U.S. Pat. No. 6,423,493, Combinatorial selection of oligonucleoside aptamers, D.Gorenstein, et al., issued Jul. 23, 2002.
U.S. Pat. No. 6,120,763, Compositions and methods for enhanced tumor cell immunity in vivo, H. Fakhrai, et al., issued Sep. 19, 2000.

What is claimed is:

1. A partially thio-modified aptamer that binds to a protein comprising RNase H activity.

2. The aptamer of claim 1, having the thioate and sequence substitutions of the oligonucleotides identified by SEQ ID NOS.: 1, 2 and the combination thereof.

3. The aptamer of claim 1, having the sequence of the formula:

| | |
|---|---|
| 5′ ATGCTTCCACGAGCCTTTCGGGGTTGGTGTₛA | (SEQ ID NO.:1) |
| CₛAGTGGₛATGGCTGCGₛAGGCGGTₛAGTCTₛATTC | |
| 3′ | |
| 3′ TAₛCGAₛAₛGGTGCTCGGAₛAₛAₛGCCCCAₛAₛ | (SEQ ID NO.:2) |
| CCAₛCAₛTGTCAₛCCTAₛCCGACGCTCCGCCATCAG | |
| ATAAG 5′. | |

4. The aptamer of claim 1, further comprising one or more pharmaceutically acceptable salts.

5. The aptamer of claim 1, further comprising a diluent.

6. The aptamer of claim 1, wherein the aptamer is achiral.

7. The aptamer of claim 1, wherein the protein comprises a Reverse Transcriptase.

8. The aptamer of claim 1, wherein the protein comprises an HIV Reverse Transcriptase.

9. The aptamer of claim 1, wherein the aptamer comprises one or more achiral thiomonophosphates.

10. The aptamer of claim 1, wherein the aptamer comprises one or more dithiophosphates.

11. A partially thio-modified aptamer that inhibits a Reverse Transcriptase.

12. The aptamer of claim 11, wherein Reverse Transcriptase comprises an HIV Reverse Transcriptase.

13. The aptamer of claim 11, wherein the aptamer comprises one or more thio-modifications as set forth in SEQ ID NOS.: 1-32.

14. The aptamer of claim 12, wherein the aptamer comprises a short interfering RNA (siRNA); a micro, interfering RNA (miRNA); a small, temporal RNA (stRNA); or a short, hairpin RNA (shRNA).

15. A method of identifying an RNase H specific-thioaptamer comprising the steps of:
synthesizing a random phosphodiester oligonucleotide combinatorial library;
contacting the partially thiophosphate-modified oligonucleotide combinatorial library with a protein having RNaseH activity; and
isolating a subset of oligonucleotides binding to the target molecule.

16. The method of claim 15, wherein the modified nucleotide comprises a phosphorothioate.

17. The method of claim 15, wherein the modified nucleotide comprises a phosphorodithioate.

18. The method of claim 15, wherein the one or more thio-modifications is selected from the group consisting of dATP(αS), dTTP(αS), dCTP(αS), dGTP(αS), rUTP (αS), rATP(αS), rCTP(αS), rGTP(αS), dATP(αS₂), dTTP(αS₂), dCTP(αS₂), dGTP(αS₂), rATP(αS₂), rCTP(αS₂), rGTP(αS₂) and rUTP(αS₂).

19. The method of claim 15, wherein no more than three adjacent phosphate sites are replaced with phosphorothioate groups.

20. The method of claim 15, wherein at least a portion of non-adjacent phosphate sites are replaced with phosphorothioate groups.

21. The method of claim 15, wherein no more than three adjacent phosphate sites are replaced with phosphorodithioate groups.

22. The method of claim 15, wherein at least a portion of non-adjacent phosphate sites are replaced with phosphorodithioate groups.

23. The method of claim 15, wherein the target is a viral protein having RNase H activity.

24. The method of claim 15, wherein the target is HIV RT.

25. A method of identifying a set of aptamers containing an optimal composition of thiophosphate-modified nucleotides such that the aptamers bind with high affinity to a protein having RNase H activity, and have increased resistance to nuclease degradation, said method comprising the steps of:
synthesizing a random partially thiophosphate-modified oligonucleotide combinatorial library wherein at least a portion of the oligonucleotide phosphate groups are thiophosphate-modified nucleotides, and where no more than three of the four different nucleotides are substituted on the 5′-phosphate positions by 5′-thiophosphates in each synthesized oligonucleotide are thiophosphate-modified nucleotides;
contacting the amplified library with the protein under conditions favorable for binding of a binding oligonucleotide with said target molecule; and
isolating a subset of binding oligonucleotides from the library, that bind with higher affinity to the protein relative to the original library.

26. The method of claim 25, further comprising the step of:
repeating the selection iteratively, whereby an enriched subset of oligonucleotides binding with higher affinity to the target molecule relative to the original amplified subset, is isolated after each cycle.

27. The method of claim 25, whereby each iteration is performed under conditions of increased stringency in the contacting step until a subset of high affinity binding oligonucleotides is identified.

28. The method of claim 25, whereby synthesis of the combinatorial library is done using constituent oligonucleotides comprising at least a set of 5′ and 3′ PCR primer nucleotide sequences flanking a randomized nucleotide sequence.

29. The method of claim 25, whereby the subset of amplified oligonucleotides is cloned and where individual thiophosphate-modified oligonucleotides that bind to the target are isolated and sequenced.

30. The method of claim 25, whereby the isolated aptamer is screened relative to its respective non-modified oligonucleotide and comprises increased affinity for the protein and increased stability with respect to nuclease degradation.

31. The method of claim 25, whereby the thiophosphate comprises a phosphorothioate, a phosphorodithioate or a combination thereof.

32. A thioaptamer that binds specifically to and inhibits RNase H activity.

33. The thioaptamer of claim 32, wherein the thioaptamer is packed into a capsule, caplet, softgel, gelcap, suppository, film, granule, gum, insert, pastille, pellet, troche, lozenge, disk, poultice or wafer.

34. The thioaptamer of claim 32, wherein thioaptamer is packaged for released within about 60 minutes.

35. The thioaptamer of claim 32, wherein thioaptamer is packaged for release of over 90% within about 60 minutes and 12 hours.

36. The thioaptamer of claim 32, wherein the aptamer is packaged with one or more of the following PVPP, Povidone, a talc and a stearate.

37. The thioaptamer of claim 32, comprising further one or more inactives.

38. The thioaptamer of claim 32, wherein the thioaptamer is lyophilized.

39. The thioaptamer of claim 32, wherein the thioaptamer is suspended for delivery intravenously, intraperitoneally, intramuscularly, subcutaneously, intracutaneously, alveolarly, sublingual or combinations thereof.

40. The thioaptamer of claim 32, having the thioate and sequence substitutions of the oligonucleotides identified by SEQ ID NOS.: 1, 2 and the combination thereof.

41. The thioaptamer of claim 32, having the sequence of the formula:

| | |
|---|---|
| 5′ ATGCTTCCACGAGCCTTTCGGGGTTGGTGTsAC | (SEQ ID NO.:1) |
| sAGTGGsATGGCTGCGsAGGCGGTsAGTCTsATTC | |
| 3′ | |
| 3′ TAsCGAsAsGGTGCTCGGAsAsAsGCCCCAsAs | (SEQ ID NO.:2) |
| CCAsCAsTGTCAsCCTAsCCGACGCTCCGCCATCAG | |
| ATAAG 5′. | |

42. The thioaptamer of claim 32, further comprising one or more pharmaceutically acceptable salts.

43. A pharmaceutical formulation in which a therapeutically effective amount of a thioaptamer that bind specifically to and inhibits RNase H activity is provided to a patient in need thereof.


SEQUENCE LISTING (2 sequences)

SEQ ID NO: 1 - DNA, 63 bp, Artificial Sequence
  Synthetic oligonucleotide
  thioate linkage (30)..(30): thioate linkage
  thioate linkage (32)..(33): thioate linkage
  thioate linkage (37)..(38): thioate linkage
  thioate linkage (46)..(47): thioate linkage
  thioate linkage (53)..(54): thioate linkage
  thioate linkage (58)..(59): thioate linkage
aatgcttccacgagcctttcggggttggtgtacagtggatggctgcgaggcggtagtctattc

SEQ ID NO: 2 - DNA, 62 bp, Artificial
  Synthetic oligonucleotide
  thioate linkage (23)..(24): thioate linkage
  thioate linkage (27)..(28): thioate linkage
  thioate linkage (32)..(33): thioate linkage
  thioate linkage (33)..(34): thioate linkage
  thioate linkage (37)..(38): thioate linkage
  thioate linkage (38)..(39): thioate linkage
  thioate linkage (44)..(45): thioate linkage
  thioate linkage (45)..(46): thioate linkage
  thioate linkage (46)..(47): thioate linkage
  thioate linkage (56)..(57): thioate linkage
  thioate linkage (57)..(58): thioate linkage
  thioate linkage (60)..(61): thioate linkage
gaatagactaccgcctcgcagccatccactgtacaccaaccccgaaaggctcgtggaagcat